(19) United States
(12) Patent Application Publication
(10) Pub. No.: US 2014/0148802 A1
(43) Pub. Date: May 29, 2014

(54) Radio Frequency Procedure Protection

(71) Applicant: Charles LeMay, Portsmouth, NH (US)

(72) Inventor: Charles LeMay, Portsmouth, NH (US)

(21) Appl. No.: 14/233,279

(22) PCT Filed: Jul. 28, 2011
(86) PCT No.: PCT/US2011/045660

§ 371 Date: Jan. 16, 2014

(51) Int. Cl. A61B 18/12

(52) U.S. Cl. CPC A61B 18/1233
USPC 606/33

(57) ABSTRACT

A method and apparatus is provided for protecting a patient during a radio frequency medical procedure. A detector includes an input receiving electrical impulses representing at least one patient parameter to be monitored from at least one shielded sensor connected to the patient. The detector detects at least one of (a) a voltage associated with parasitic current generated during a radio frequency medical procedure and (b) a current generated during a radio frequency medical procedure. A control circuit is coupled to the detector and compares at least one of (a) the detected voltage with a threshold voltage value and (b) the detected current with a threshold current value. A switch is coupled between the detector and the control circuit. In response to a control signal generated by the control circuit, the switch selectively switches between a conductive state and a non conductive state. The conductive state maintains a low impedance at the at least one shielded sensor and maintains the shield of the at least one shielded sensor in an effective state when at least one of (a) the detected voltage is below the threshold voltage value and (b) the detected current is below the threshold current value. The non-conductive state provides a high impedance at the at least one shielded sensor and causes the shield of the at least one shielded sensor to become ineffective when at least one of (a) the detected voltage is equal to or greater than the threshold voltage value and (b) the detected current is equal to or greater than the threshold current.

### FIELD OF THE INVENTION

This invention concerns a system and method for protecting patients and patient monitoring equipment during a medical procedure utilizing radio frequency energy such as electrosurgery.

### BACKGROUND OF THE INVENTION

During a radio frequency procedure such as an electrosurgical procedure, a high-frequency electric current (AC current) is applied to biological tissue in order to directly heat the biological tissue to treat a patient for a particular condition. Electrosurgical devices beneficially enable a practitioner to treat a subject area with precision while reducing an amount of blood loss from the patient undergoing treatment. The electrosurgical instrument may include an electrosurgical generator that generates a voltage which is applied through an electrosurgical tool to the patient. The patient's body is contacted by a return plate which receives the current flowing through the patient enabling the return to ground. The return plate may be a relatively large metal plate or a flexible metalized plastic pad which is connected to the return electrode of the AC source. During the procedure, the electric current flows from the active electrode on the electrosurgical tool, through the body to the return electrode, and then back to the electrosurgical generator.

As this is a surgical procedure, there is a recognized need to monitor a plurality of patient parameters during the surgery to insure that the patient vitals are stable. For example, conventional monitoring may include an electrocardiogram (ECG) monitor that is connected to the patient by a plurality of leads that monitor the electrical impulses of the patient's heart. However, a problem exists whereby a patient burn may be caused by the increased current present during an electrosurgical procedure. Moreover, in order to prevent a burn, the insertion of a high impedance block significantly degrades the quality of the signal being monitored by the ECG. An exemplary block diagram showing the conventional method of protecting a patient from being burned is shown in FIG. 1. An ECG monitor 12 is shown connected via at least one electrical lead 14 to a patient 11. A capacitance 16 is present between the lead 14 and the shield, which is in turn connected to an inductor 18 that provides an impedance sufficient to prevent the patient from being burned during an electrosurgical procedure. A significant drawback with the system shown in FIG. 1 is that the impedance 18 is typically an external block that must be connected prior to the electrosurgical procedure and removed after the procedure is completed. This block may include at least one choke placed in the ECG shield leads to act as the high impedance to the RF (radio frequency) energy created during electrosurgery. This results in a medical professional having to compromise with respect to monitoring the patient in order to protect the patient during electrosurgery because the inadequate shielding due to the use of the external block causes a degradation of ECG signal quality.

While conventional burn prevention systems are able to provide the necessary impedances to prevent burning a patient, these systems result in degraded monitoring. A system according to invention principles addresses deficiencies of known systems.

### SUMMARY OF THE INVENTION

In one embodiment, an apparatus is provided for protecting a patient during a radio frequency medical procedure. A detector includes an input receiving electrical impulses representing at least one patient parameter to be monitored from at least one shielded sensor connected to the patient. The detector detects at least one of (a) a voltage associated with parasitic current generated during a radio frequency medical procedure and (b) a current generated during a radio frequency medical procedure. A control circuit is coupled to the detector and compares at least one of (a) the detected voltage with a threshold voltage value and (b) the detected current with a threshold current value. A switch is coupled between the detector and the control circuit. In response to a control signal generated by the control circuit, the switch selectively switches between a conductive state and a non conductive state. The conductive state maintains a low impedance at the at least one shielded sensor and maintains the shield of the at least one shielded sensor in an effective state when at least one of (a) the detected voltage is below the threshold voltage value and (b) the detected current is below the threshold current value. The non-conductive state provides a high impedance at the at least one shielded sensor and causes the shield of the at least one shielded sensor to become ineffective when at least one of (a) the detected voltage is equal to or greater than the threshold voltage value and (b) the detected current is equal to or greater than the threshold current.

In another embodiment, a method is provided for protecting a patient during a radio frequency medical procedure. The method of protecting a patient includes detecting at least one of (a) voltage associated with a parasitic current generated during an radio frequency medical procedure and (b) a current generated during a radio frequency medical procedure, at an input that receives electrical impulses representing at least one patient parameter to be monitored from at least one shielded sensor connected to the patient. A control circuit compares at least one of (a) the voltage detected by the detector with a threshold voltage value and (b) the current detected by the detector with a threshold current value. In response to a control signal generated by the control circuit, a switch switches between a conductive state and a non-conductive state. The conductive state maintains a low impedance at the at least one shielded sensor and maintaining the shield in an effective state when at least one of (a) the detected voltage is below the threshold voltage value and (b) the detected current is below the threshold current value. The non-conductive state provides a high impedance at the at least one shielded sensor and causes the shield of the sensor to become ineffective when at least one of (a) the detected voltage is equal to or greater than the threshold voltage value and (b) the detected current is equal to or greater than the threshold current value.

### BRIEF DESCRIPTION OF THE DRAWINGS

FIG. 1 depicts a prior art setup of patient protection device;

FIG. 2 depicts an exemplary setup of a patient undergoing an electrosurgical procedure;

FIG. 3 is an exemplary block diagram of a patient protection apparatus according to invention principles;

FIG. 4 is an exemplary block diagram of a patient protection apparatus according to invention principles;

FIG. 5 is an exemplary block diagram of a control circuit for a patient protection apparatus according to invention principles;

FIG. 6 is an exemplary circuit diagram of a patient protection apparatus according to invention principles;

FIGS. 7A and 7B are graphical representations of the operation of the patient protection apparatus according to invention principles; and

FIG. 8 is a flow diagram detailing the operation of the patient protection apparatus according to invention principles.

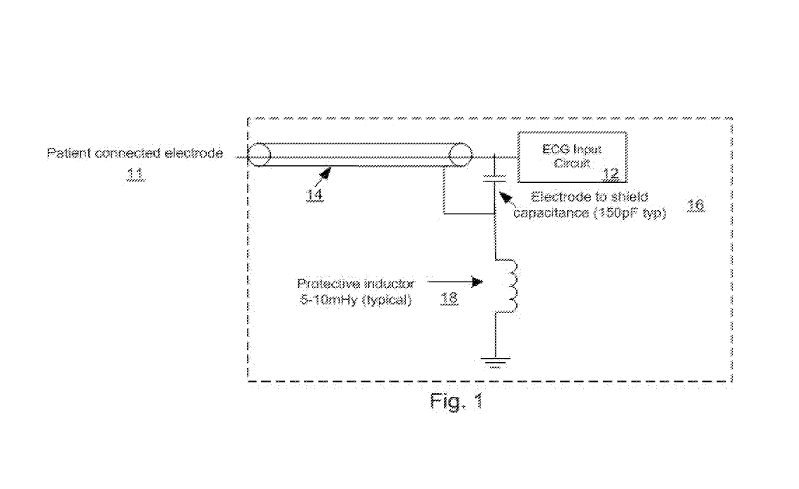

### DETAILED DESCRIPTION

The patient protection apparatus automatically increases an impedance of patient connected ECG leads. This advantageously provides a shield during a medical procedure employing RF energy. A medical procedure employing RF energy generates an electromagnetic frequency and/or RF current that is applied to tissue of a patient. Examples of medical proceduces using RF energry to treat a patient includes but is not limited to (a) electrosurgery; (b) RF ablation; (c) RF lesioning; (d) any other RF engery application to tissue. The patient protection apparatus advantageously provides a shield during electrosurgery to reduce energy conduction and thus prevent patient burns by automatically detecting an increased amount of current generated by an active electrosurgical tool. An electrosurgical procedure is one type of medical procedure employing RF energy and is described for purposes of example only. One skilled in the art would recognize the ability to employ the patient protection apparatus during any RF procedure thereby increasing the impedance of a patient connected to a monitoring device. A current burn to a patient may be considered a function of electrode surface area and the current density flowing through the electrode. Thus, the apparatus may be able to effectively provide a high impedance and prevent burns at the site of the electrode on a patient's body. The apparatus also advantageously returns the impedance and the ECG shields to a low impedance level when an amount of current flowing through the body of the patient is below a threshold value indicating that there is no electrosurgical procedure currently underway. Thus, the automatic reversion to a low impedance condition advantageously provides a high level of shielding and consequently high signal integrity. Therefore, the patient protection apparatus advantageously provides instantaneous and automatic mode changes between good patient protection on the one hand and high performance ECG monitoring (low noise, good bandwidth) on the other.

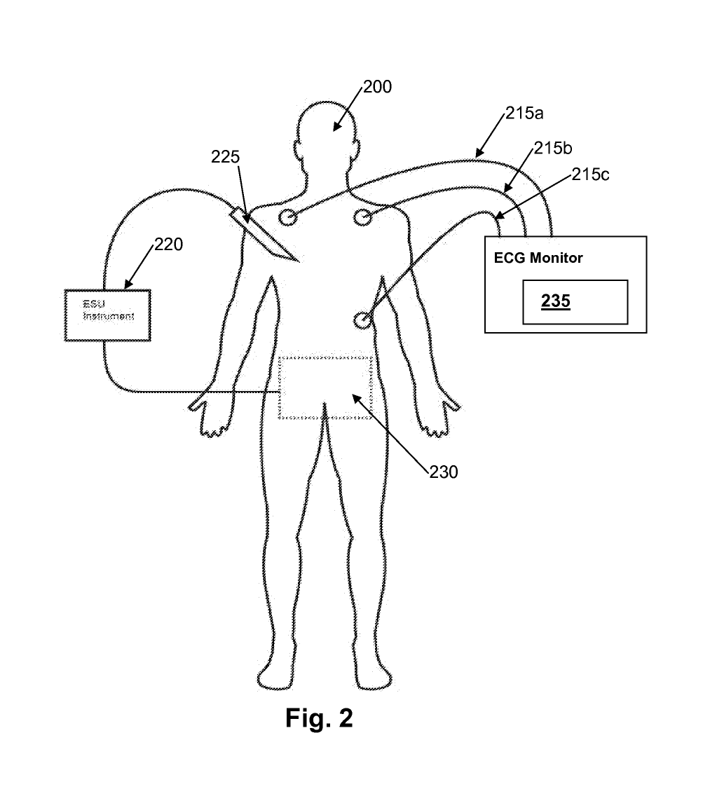

FIG. 2 is a schematic diagram detailing the operation of the patient protection apparatus in a clinical situation. Shown herein is a patient 200 undergoing an medical procedure using RF energy. As shown herein and in the following figures, the RF procedure is an electrosurgical procedure. The patient 200 is being monitored by a patient parameter monitor 210. The patient parameter monitor 210 is connected to the patient by a plurality of electrical leads 215a-215c. The patient parameter monitor may sense electrical impulses in the patient and automatically convert the sensed impulses into medically relevant data to be presented to a medical professional. An exemplary patient parameter monitor 210 is an ECG monitor. However, the patient protection apparatus may be included in any patient parameter monitor 210 able to sense electrical impulses.

During electrosurgery, the patient 200 is connected to an ECG monitor in the conventional manner as discussed above. An electrosurgical apparatus includes an electrosurgical generator 220 electrically connected between an electrosurgical tool 225 and a return plate 230. The electrosurgical tool may be any type of electrosurgical tool that is medically appropriate for the surgery being performed and to treat the patient 200. In one embodiment, the electrosurgical tool 225 is an electric scalpel which applies high energy RF power that is generated by the generator 220. The high energy RF power is applied to a precise point on the patient and the current flowing to the tool 225 typically returns to the generator 220 through the return plate 230 thereby completing the electrosurgical circuit.

Depending on the type of electrosurgical procedure being performed and the location at which the electrosurgical tool 225 is operating on the patient's body 200, placement of the electrical leads 215a-215c may result in a low impedance unintended pathway for current flowing through the electrosurgical tool 225. For example, in the instance where the patient parameter monitor 210 is an ECG monitor and the electrical leads 215a-215c are ECG leads connected to the patient 200, the placement of the ECG electrodes in relation to the electrosurgical site (e.g. the point at which the tool contacts the patient's body) results in a low impedance pathway for the current. Should this low impedance pathway be formed, the current applied by the electrosurgical tool 225 gets shunted away from the patient's body 200 and instead flows through the ECG leads resulting in current density being present at the ECG electrode that increases the temperature, causing a burn to the skin of the patient 200 at the point where the ECG electrodes are attached. This increased current density occurs because the individual electrodes cover a small area on the patient 200.

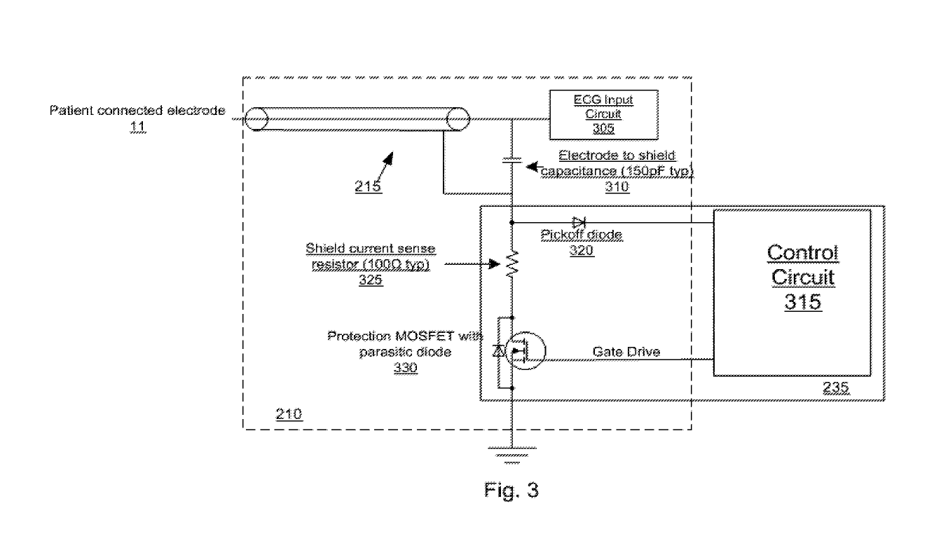

A patient protection apparatus 235, which will be described in greater detail below with respect to FIGS. 3-8, is connected within the patient parameter monitor 210 and automatically senses a level of current that is flowing through the electrode shields at any given time. The apparatus 235 automatically switches operational modes depending on the amount of current sensed. In a first mode of operation, the apparatus senses a current flowing through the patient electrode is within a first range indicating that risk of patient burn is low. This mode of operation corresponds to a period of time when no electrosurgical procedure is being performed. In this first mode of operation, shields on the leads 215a-215c are maintained in a low impedance state providing a high degree of shielding while ensuring that the patient parameter signal being monitored is of a high quality. The apparatus 235 automatically switches to a second mode of operation when the apparatus senses that the current flowing through the shield of a respective electrode reaches a threshold current level (e.g. 10 mA). Sensing that the current flowing through the shield is equal to or greater than the threshold current value, the circuit is triggered and the second mode of operation begins. In the second mode of operation, the apparatus 235 is no longer monitoring the current but instead monitors the voltage on the now high impedance shield. In the second mode of operation, the apparatus 235 increases the impedance in the leads thereby reducing the current flowing through the shields and protecting the patient from being burned. The patient protection apparatus 235 advantageously enables instantaneous switching between the modes of operation thereby improving a level of protection to the patient from a burn hazard while simultaneously improving the quality of an ECG signal between applications of the ESU electrosurgical tool.

FIG. 3 is an exemplary circuit diagram of the patient protection apparatus 235 embodied in a patient parameter monitoring device 210. The patient monitoring device 210 includes a monitoring input circuit 305 coupled to receive an input signal from to at least one monitoring lead 215 connected to the patient via an electrode (not shown). The monitoring input circuit may sense an electrical signal representing a patient parameter transmitted from the patient via the electrode and the monitoring lead 215. In one embodiment, the monitoring device 210 is an ECG monitor and the at least one lead 215 is an ECG monitoring lead having an electrode that is connected to the patient to monitor cardiac impulses of the patient. In another embodiment, the monitoring input circuit may be a temperature monitoring circuit that selectively monitors patient temperature. While only a single monitoring lead 215 is shown, one skilled in the art will recognize that any number of monitoring leads 215 may be connected to the input circuit 305 for monitoring the desired patient parameter. A capacitance 310 is present between the monitoring lead 215 and provides a low impedance shield which increases the quality of the signal being monitored by the monitoring device 210. In one embodiment the capacitance 310 provides a capacitance of substantially 150 pF. The capacitance may not be a discrete capacitor and instead is a parasitic capacitance formed by the geometry of the coaxial construction of the cable carrying the electrode.

The patient protection apparatus 235 includes a control circuit 315 that senses parasitic current in the shield and determines that a patient burn potential exists. At that time, the apparatus 235 switches from the first mode of operation to the second mode in the manner discussed below. The patient protection apparatus 235 includes at least one resistor 325 connected between a pick-off diode 320 and a switch 330. In one embodiment, the switch 330 is a MOSFET switch. In another embodiment, the switch 330 is a MOSFET switch that has an inherent internal resistance such that there need not be a discrete resistor present in the apparatus. The control circuit 315 is also connected between the pick-off diode 325 and the switch 330 thereby forming a complete circuit when the switch 330 is a first “on” position. When the switch 330 is in the first “on” position, the switch 330 is conducting, providing a low impedance shield for the particular electrode enabling high quality monitoring of an input signal by the monitoring circuit 305.

In the event that parasitic current is conducted along the lead 215, a voltage is created across the at least one resistor 325 and the control circuit 315 automatically senses this voltage via the pick-off diode 320. Upon sensing of this voltage across the resistor 325, the control circuit causes the switch to move from the first “on” position to the second “off” position. In the second “off” position, the switch 330 is not conducting and the shield is allowed to float and move along in voltage with the signal conductor of the input cable (e.g. the center lead of a coaxial cable). This prevents the parasitic current from flowing through the shield and burning the patient at the point which the electrode is connected to the patient. When the switch 330 is in the second “off” position, the quality of the signal being monitored by the monitoring circuit 305 is reduced but the patient is protected which is a necessary trade-off that must be made during an electrosurgical procedure. However, the patient protection apparatus 235 advantageously reduces an amount of time that the quality is reduced because the protection is enabled in response to the voltage sensed at resistor 325.

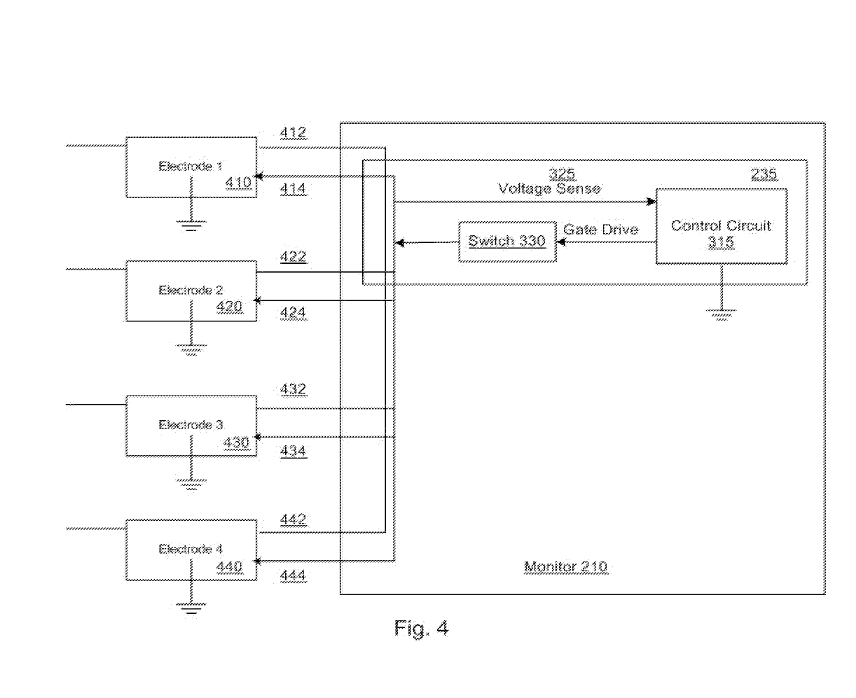

FIG. 4 is an exemplary circuit diagram of a patient parameter monitor 210 including the patient protection apparatus 235. In this embodiment, a plurality of electrodes 410, 420, 430 and 440 are connected to the monitoring device 210. Electrode 410 is connected to the monitoring device 210 by an input line 412 and an output line 414. Electrode 420 is similarly connected to the monitoring device 210 by an input line 422 and an output line 424. Electrode 430 is connected to the monitoring device 210 by input line 432 and output line 434 and electrode 440 is connected to the monitoring device by input line 442 and output line 444.

The patient protection apparatus 235 includes the at least one resistor 320, the pick-off diode 325 and the switch 330 (see FIG. 3). The respective input lines 412, 422, 432 and 442 are electrically connected to the pickoff diode 325 and the respective output lines 414, 424, 434 and 444 are electrically connected to the switch 330. The control circuit 315 controls the operation of the switch 300 and is responsive to a voltage sensed at the pickoff diode 325. In the first mode of operation, the switch 330 is in the “on” position and the shield for each electrode 410, 420, 430 and 440 are held in a low impedance state enabling high quality signal monitoring.

The control circuit 315 automatically changes the position of the switch 330 in response to introduction of parasitic current at any one of the electrodes 410, 420, 430 and/or 440. Parasitic current may result during an electrosurgical procedure which may cause a voltage to be generated across the resistor 320 and sensed by the pickoff diode 325. If the sensed voltage is above a threshold value (greater than a reference voltage in the order of 1 Volt), the control circuit causes the switch to move from the first “on” position to the second “off” position. When this occurs, the control circuit 315 turns “off” the switch 330, releasing the shields and reducing the current dramatically. Because the switch 330 is no longer sinking significant current, the voltage on the shield sensed by the control circuit 315 now follows the voltage on the patient electrode thereby maintaining the position of the switch 330 in the second “off” position. When the electrosurgical procedure ends or, in the event that the medical professional pauses the procedure by deactivating the electrosurgical tool, the voltage drops across the resistor 320, the pickoff diode 325 senses the voltage change and the control circuit 315, and the switch 330 returns to the first “on” position rendering the shields on the respective electrodes effective and enabling high quality patient parameter monitoring.

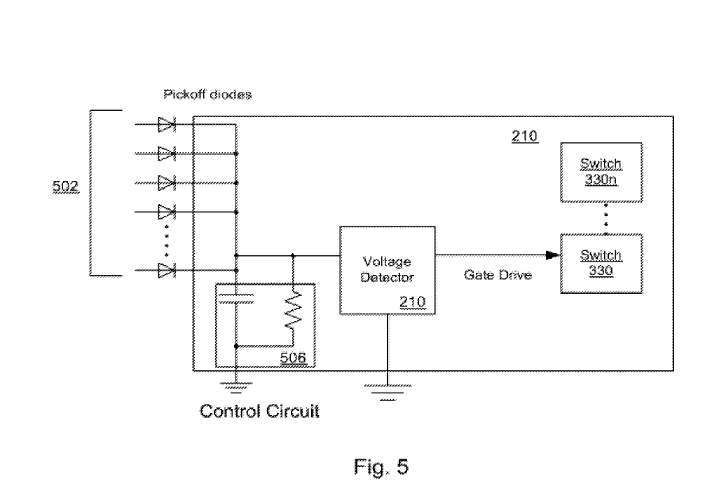

FIG. 5 is an exemplary circuit diagram of the control circuit 315. The control circuit 315 receives current from a plurality of pickoff diodes represented collectively by reference numeral 502. One skilled in the art will recognize that the number of pickoff diodes 502 is equal to the number of electrodes that are connected to the patient through which a patient parameter signal is able to be monitored by a monitoring device. In one embodiment, the patient parameter monitor is an ECG monitor and there are four ECG leads, each including a respective electrode connected to the patient for monitoring electrical impulses representing cardiac data.

Under conditions of high current induced in the shields, current enters through the most positive electrode, passes through resistor 320 to “ground”, while at the corresponding most negative electrode, the said current passes through the parasitic diode in the MOSFET switch, through the resistor 320 for that second electrode, and back through that shield to the patient. Simultaneously, the most positive electrode drives the corresponding pickoff diode 325 into conduction, charging an input capacitor 506 (peak detector) in the control circuit 315. A voltage detector 504 of the control circuit is coupled to the switch 330 and directs the operation of the switch between the first “on” position and the second “off” position. In one embodiment, the voltage detector 504 may be a comparator which compares the voltage sensed by the peak detector 506 to a threshold value and, if the sensed voltage is equal to or greater than the threshold, the switch is caused to move from the first “on” position to the second “off” position. When the switch is in the “off” position, the impedance level is increased, thereby disabling any shield on the electrode and substantially reducing the current from flowing through the electrode and thus preventing a patient burn. The time constant of the circuit determines the reset time of the circuit (typically milliseconds). This time constant is effectively the “recovery” time of the circuit, and corresponds to the time when the shields are again effective.

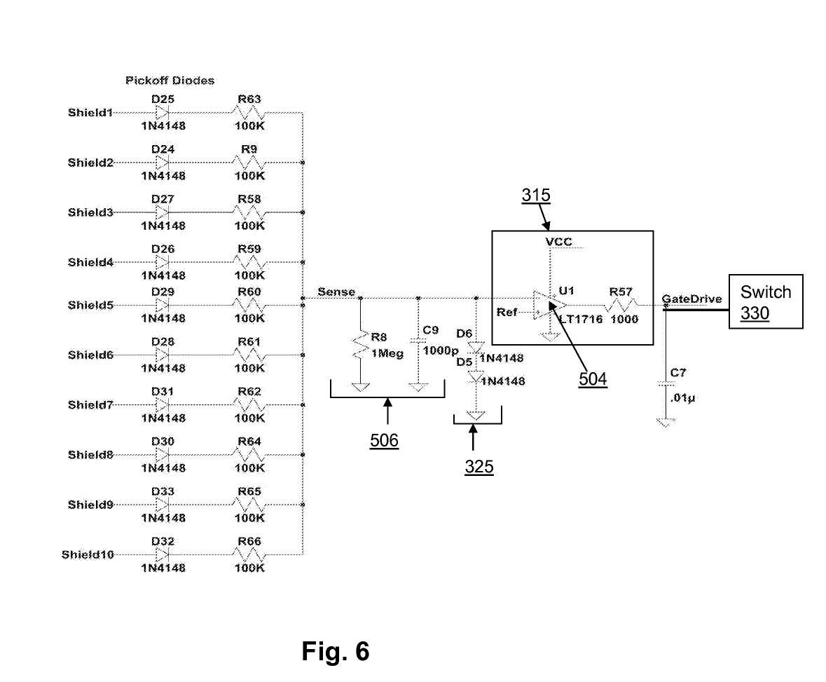

FIG. 6 is an exemplary circuit diagram of a plurality of electrical leads that may be connected to a patient for monitoring at least one patient parameter. Each lead includes a shield, shown herein as Shield1-Shield 10, which provides a low impedance during patient monitoring. Each of Shield 1-Shield 10 is connected to a respective pickoff diode and any parasitic current flowing through any of the respective leads and pickoff diode is detected by the peak detector 506. Peak detector 506 comprises a resistor and a capacitor. Voltage detector 504 continuously compares the voltage detected across the peak detector 506 with a threshold voltage and controls the switch 330 to move between the “on” position and “off” position.

During electrosurgery, current flows through the patient and further through the leads connecting the patient monitor to the patient. During this procedure, one lead has more current flowing therethrough and is hotter than the other leads. Current flows down this lead which is provided to the at least one resistor and the switch (e.g. MOSFET). The current on the shield flows through the resistor resulting in a voltage being sensed by the pickoff diode associated therewith thereby resulting in the opening of the switch to the “off position”. The peak detector 506 is charged and the voltage detector 504 recognizes the voltage resulting from the current in the shield reached the threshold value and automatically switches switch from the “on” position into the “off” position. In the “off” position, the shields are able to float thereby increasing the impedance on the lead and preventing the current density at the respective electrodes from increasing and causing a burn on the skin of the patient. When the voltage detector determines that the voltage is below the threshold value, the control circuit 315 causes the switch to return to the “on” position and begin conducting again thereby lowering the impedance and improving the signal quality being monitored by the patient monitoring device.

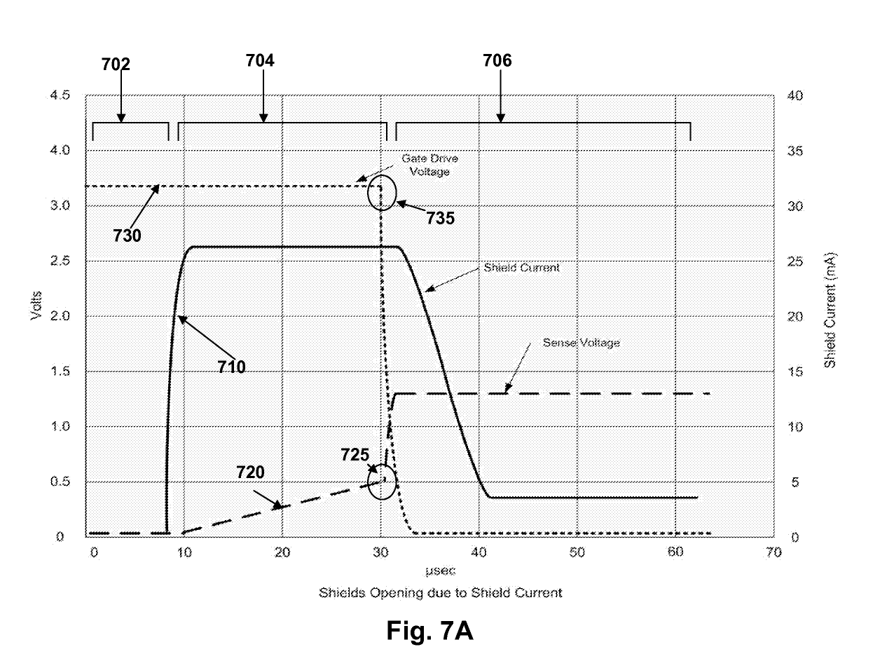

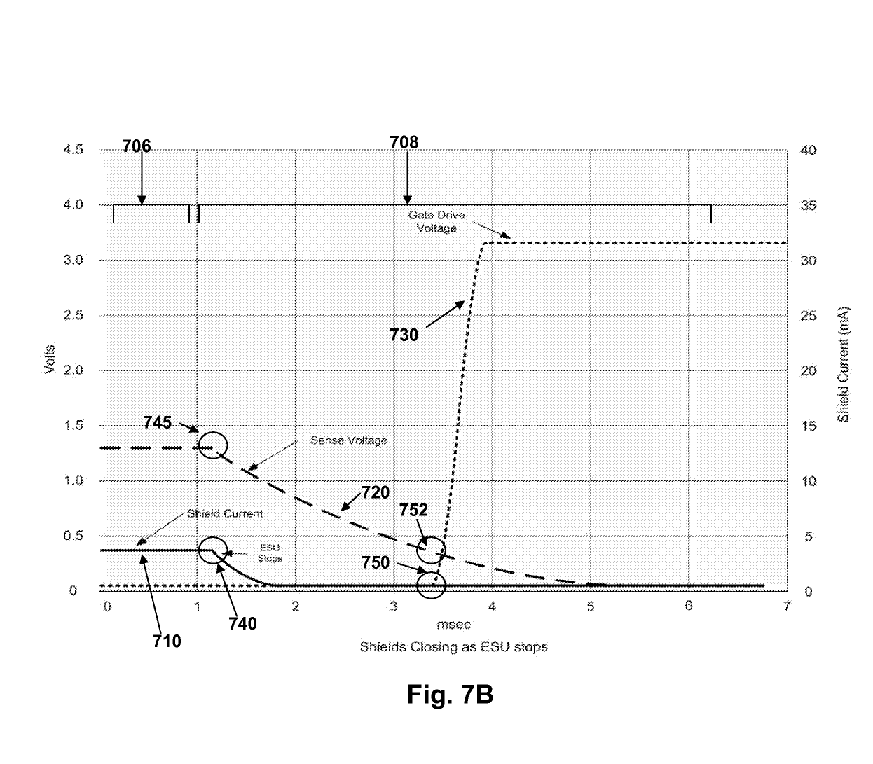

FIGS. 7A and 7B are graphical representations showing the voltages present prior to, during and after an electrosurgical procedure which detail the operation of the patient protection apparatus connected to an ECG monitoring device. Alternatively, this may represent an ECG monitoring device having the patient protection apparatus formed integral therewith. FIGS. 7A and 7B include three plots representing the current through the resistor 710, voltage detected by the voltage detector 720 and control voltage to the MOSFET switch 730. These plots are shown at various times including prior to an electrosurgical procedure 702, initiation of an electrosurgical procedure 704, during the electrosurgical procedure 706 and post-electrosurgical procedure 708.

Referring now to FIG. 7A, prior to the electrosurgical procedure 702, the voltage across the voltage detector on plot 720 and the gate voltage to the MOSFET switch 730 are substantially flat and constant. This results in a low impedance across the ECG leads that results in a high quality signal being monitored from the patient. At the initiation of an electrosurgical event 704, the current in the resistor reaches a threshold value of substantially 27 mA in less than microseconds as seen from plot 710. This increased current is sensed by a pickoff diode and charges the capacitor of the peak detector and the voltage detector detects a voltage above the threshold voltage at point 725. At this time, the control circuit drives the gate of the MOSFET low causing the MOSFET switch to move from the first “on” position to the second “off” position (point 735) which “releases” the shield, which in turn rapidly drives the sensed voltage up to the 1.2V clamp. It should be noted that points 725 and 735 occur at substantially the same time during the timeline. The result is an increased impedance on the leads which results in a decrease in current density at one or more electrodes.

An advantageous feature of the patient protection apparatus is that the circuit switches abruptly from the first “on” position to the second “off” position as a result of sensing the shield current as the ESU procedure begins, and subsequently switching from the second “off” position the first “on” position as a result of sensing the shield voltage during the ESU procedure through the time when the ESU procedure ends. This helps prevent the circuit from oscillating on and off at the edge of operation.

The electrosurgical procedure continues during the time represented by reference numeral 706. During this time period, there is an increase in voltage sensed by the voltage detector due to the floating shield. The voltage detector continuously compares the sensed voltage to the threshold and MOSFET switch operation is controlled thereby. During the electrosurgical procedure the current flowing through the resistor is substantially decreased (approximately 3 mA) and remains substantially constant through the procedure.

Referring now to FIG. 7B which is a continuation of the plots 710, 720 and 730 of FIG. 7A, reference numeral 706 indicates that the electrosurgical procedure is continuing. The conclusion of the electrosurgical event is represented by reference numeral 708 and begins at point 740 on plot 710 and point 745 on plot 720 whereby an amount of current flowing through the resistor returns to the near zero level seen during pre-electrosurgical procedure 702. As shown in plots 710 and 720 of FIG. 7B, the current through the resistor decreases along with the voltage sensed by the voltage detector. When the voltage sensed by the voltage detector falls below a threshold value as indicated by point 752 on plot 720, the control circuit automatically causes the MOSFET to move from the “off” position to the “on” position, as indicated by point 750 on plot 730. When in the on position, the MOSFET conducts and places the shields in a low impedance state thereby enabling high quality monitoring of a particular patient parameter.

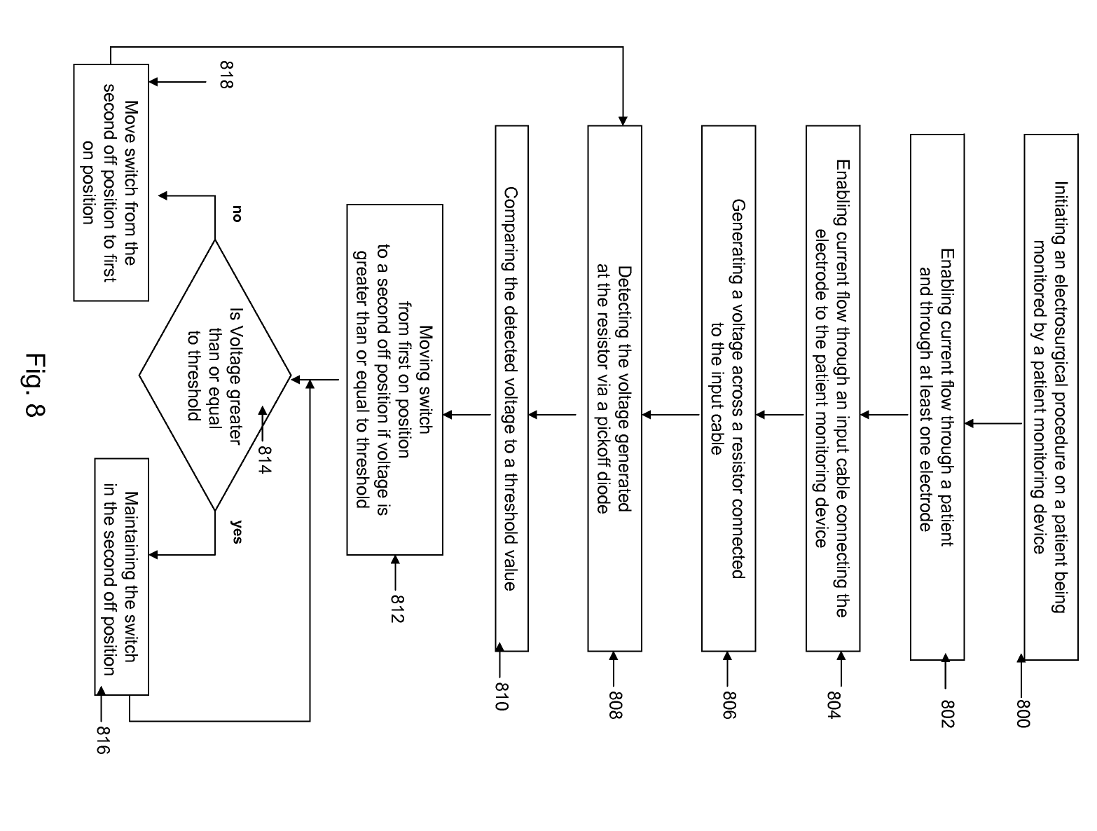

The operation of the patient protection apparatus is now described with respect to the flowchart of FIG. 8. In step 800, an electrosurgical procedure is initiated on a patient being monitored by a patient monitoring device. The initiation of the electrosurgical procedure may include using an electrosurgical tool to apply RF energy to a point on the patient for a medically relevant purpose to provide treatment to the patient. The application of RF energy to the patient results in current flowing through the patient to a return plate and back to the electrosurgical generator. In step 802, the current flowing through the patient also flows through at least one electrode connecting the patient to the patient monitoring device. The current flows through the electrode and over an input cable that connects the electrode to the patient monitor in step 804. A voltage corresponding to the current flowing through the input cable is generated at a resistor in step 806 and in step 808, a pickoff diode detects the voltage generated across the resistor in step 806. A voltage detector in a control circuit compares the detected voltage to a threshold value in step 810. If the detected voltage is equal to or greater than a threshold value, the control circuit causes a MOSFET switch to move from a first “on” position to a second “off” position. When in the second “off” position, the MOSFET is in a non-conducting state and substantially decreases in current density at the one or more electrodes connecting the patient to the patient monitoring device as stated in step 812. The voltage detector continuously determines if the sensed voltage is equal to or greater than the threshold in step 814. If the determination is positive, the switch remains in the “off” position as indicated in step 816. If the determination in step 814 is negative, then it is indicative of the end of an electrosurgical procedure and the control circuit causes the MOSFET to move from the second “off” position back to the first “on” position as in step 818. In the “on” position, the switch is in a conductive state and provides a low impedance at the electrode shield which enables the signal being monitored to be of a high quality.

The patient protection apparatus described above with respect to FIGS. 2-8 may be formed integral with the patient monitoring devices and thereby enable rapid and near instantaneous switching between patient monitoring and an electrosurgical procedure. This advantageously reduces the time typically associated with inserting the inductor block that is typically used to prevent patient burns in conventional ESU procedures. Additionally, the patient protection device may be formed as a non-removable adapter that is selectively connected between the inputs of the monitoring leads and the monitoring device. Thus, the patient protection apparatus provides a relatively high level of protection to the patient from a burn hazard while simultaneously providing an ECG signal of relatively high quality between applications of the ESU electrosurgical tool.

Although the invention has been described in terms of exemplary embodiments, it is not limited thereto. Rather, the appended claims should be construed broadly to include other variants and embodiments of the invention which may be made by those skilled in the art without departing from the scope and range of equivalents of the invention. This disclosure is intended to cover any adaptations or variations of the embodiments discussed herein.

What is claimed is:

1. An apparatus for protecting a patient during a radio frequency medical procedure comprising:
a detector having an input receiving electrical impulses representing at least one patient parameter to be monitored from at least one shielded sensor connected to the patient, said detector detects at least one of (a) a voltage associated with parasitic current generated during the radio frequency medical procedure and (b) a current generated during the radio frequency medical procedure;
a control circuit, coupled to the detector, that compares at least one of (a) the detected voltage with a threshold voltage value and (b) the detected current with a threshold current value; and
a switch, coupled between the detector and the control circuit, in response to a control signal generated by the control circuit, selectively switches between
a conductive state maintaining a low impedance at the at least one shielded sensor and the shield of the at least one shielded sensor in an effective state when at least one of (a) the detected voltage is below the threshold voltage value and (b) the detected current is below the threshold current value, and
a non-conductive state providing a high impedance at the at least one shielded sensor and causes the shield of the at least one shielded sensor to become ineffective when at least one of (a) the detected voltage is equal to or greater than the threshold voltage value and (b) the detected current is equal to or greater than the threshold current.

2. The apparatus as recited in claim 1, wherein
the radio frequency medical procedure is an electrosurgical procedure.

3. The apparatus as recited in claim 1, wherein
the radio frequency medical procure is at least one of (a) a medical procedure employing radio frequency energy to generate an electromagnetic frequency and/or RF current that is applied to tissue of a patient; (b) radio frequency ablation and (b) radio frequency lesioning.

4. The apparatus as recited in claim 1, wherein
said detector includes at least one resistor that receives the parasitic current and a pickoff diode that is activated in response to a voltage differential generated across the resistor.

5. The apparatus as recited in claim 1, wherein
said switch is a MOSFET switch.

6. The apparatus as recited in claim 1, wherein
when the switch is in the non-conductive state an increase in current density is prevented from forming at the shielded sensor and burning the patient.

7. The apparatus as recited in claim 1, wherein
when the switch is in the non-conductive state, a current density is reduced at the shielded sensor and prevents the patient from being burned.

8. The apparatus as recited in claim 1, wherein
said control circuit continuously monitors detected voltages.

9. The apparatus as recited in claim 1, wherein
said apparatus is formed integral with a patient monitoring device that selectively monitors electrical impulses of a patient using the at least one shielded sensor, each of the at least one shielded sensors an electrode and is connected to the patient monitoring device, said apparatus being connected between the at least one shielded sensor and a monitoring circuit of the patient monitoring device.

10. The apparatus as recited in claim 1, wherein
said apparatus is an adapter that is selectively connected to a patient monitoring device, said adapter including a first coupling that enables connection to the at least one shielded sensor and a second coupling that enables connection of the apparatus with the patient monitoring device.

11. A method of protecting a patient during a radio frequency medical procedure comprising the activities of:
detecting at least one of (a) voltage associated with a parasitic current generated during the radio frequency medical procedure and (b) a current generated during the radio frequency medical procedure, at an input that receives electrical impulses representing at least one patient parameter to be monitored from at least one shielded sensor connected to the patient;
comparing, by a control circuit, at least one of (a) the voltage detected by the detector with a threshold voltage value and (b) the current detected by the detector with a threshold current value;
switching, in response to a control signal generated by the control circuit, between
a conductive state maintaining a low impedance at the at least one shielded sensor and maintaining the shield in an effective state when at least one of (a) the detected voltage is below the threshold voltage value and (b) the detected current is below the threshold current value, and
a non-conductive state providing a high impedance at the at least one shielded sensor and causing the shield of the sensor to become ineffective when at least one of (a) the detected voltage is equal to or greater than the threshold voltage value and (b) the detected current is equal to or greater than the threshold current value.

12. The method as recited in claim 11, wherein
the radio frequency medical procedure is an electrosurgical procedure.

13. The method as recited in claim 11, wherein
the radio frequency medical procure is at least one of (a) a medical procedure employing radio frequency energy to generate an electromagnetic frequency and/or RF current that is applied to tissue of a patient; (b) radio frequency ablation and (b) radio frequency lesioning.

14. The method as recited in claim 11, wherein
said activity of detecting includes receiving the parasitic current at a resistor and activating a pickoff diode in response to a voltage differential generated across the resistor.

15. The apparatus as recited in claim 11, wherein
said activity of switching is performed using a MOSFET switch.

16. The method as recited in claim 11, further comprising the activity of
preventing an increase in current density at the shielded sensor when in the non-conductive state.

17. The method as recited in claim 11, further comprising the activity of
reducing current density at the shielded sensor when in the non-conductive state.

18. The method as recited in claim 11, further comprising the activity of:
continuously monitoring detected voltages.

19. The method as recited in claim 11, wherein
said method is performed within a patient monitoring device that selectively monitors electrical impulses of a patient using the at least one shielded sensor, each of the at least one shielded sensor having an electrode and connected to the patient monitoring device.

20. The method as recited in claim 11, wherein
said method is performed in an adapter that is selectively connected to a patient monitoring device, the adapter including a first coupling that enables connection to the at least one shielded sensor and a second coupling that enables connection of the apparatus with the patient monitoring device.
